(19) United States
(12) Patent Application Publication
(10) Pub. No.: US 2015/0322108 A1
(43) Pub. Date: Nov. 12, 2015

(54) CRYSTALLINE POLYMORPHS

(71) Applicant: AbbVie Inc., North Chicago, IL (US)

(72) Inventors: Colleen C. Garrett, Chicago, IL (US); Lars Fredrik Nordstroem, Evanston, IL (US); Donghua Zhu, Vernon Hills, IL (US)

(21) Appl. No.: 14/274,309

(22) Filed: May 9, 2014

(51) Int. Cl. C07K 5/062

(52) U.S. Cl. CPC C07K 5/06052

(57) ABSTRACT

The present invention features crystalline forms of Compound I.

### FIELD OF THE INVENTION

The present invention relates to crystalline polymorphs of Compound I, compositions comprising the same, pharmaceutical compositions comprising the same, and methods of using the same to prepare composition and pharmaceutical compositions.

### BACKGROUND

The hepatitis C virus (HCV) is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often inadequate. Therefore, there is a need for new drugs to treat HCV infection.

### BRIEF DESCRIPTION OF THE DRAWINGS

FIG. 1 shows the powder X-ray diffraction pattern (PXRD) of Hydrate B.

FIG. 2 is a TGA-MS curve displaying weight loss upon heating of Hydrate B.

### DETAILED DESCRIPTION

The present invention features crystalline polymorphs of comprising dimethyl (2S,2′S)-1,1′-((2S,2′S)-2,2′-(4,4′-((2S,5S)-1-(4-tert-butylphenyl)pyrrolidine-2,5-diyl)bis(4,1-phenylene))bis(azanediyl)bis(oxomethylene)bis(pyrrolidine-2,1-diyl))bis(3-methyl-1-oxobutane-2,1-diyl)dicarbamate (

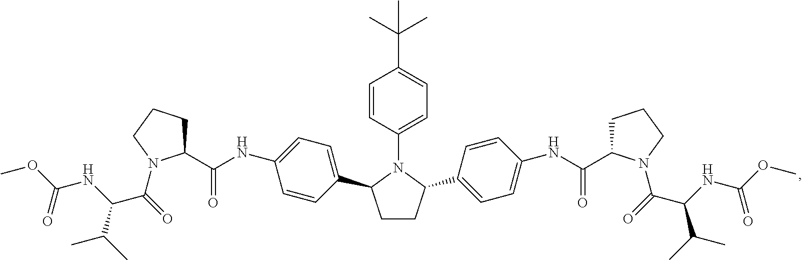

hereinafter Compound I). Compound I is a potent HCV inhibitor.

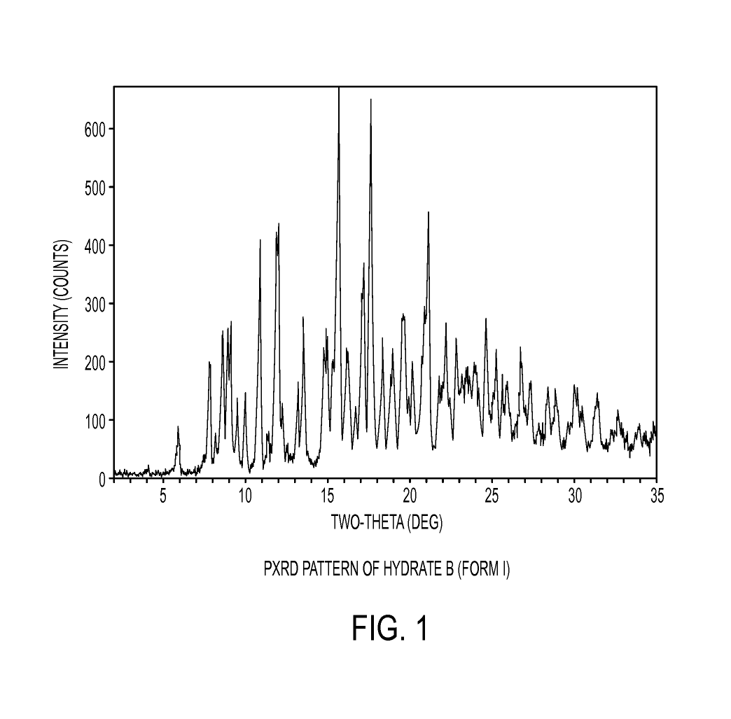

In one aspect, the present invention features a crystalline form of Compound I, which is hereinafter referred to as “Hydrate B” or “Form I”. The powder X-ray diffraction pattern (PXRD) of Hydrate B is shown in FIG. 1.

Hydrate B has characteristic peaks in the powder X-ray diffraction pattern at values of two theta (° 2θ) of 5.9, 8.6, 9.1, 10.9, 11.9, 13.5, 14.8, 15.7, 17.2, 17.7, and 19.6.

Preferably, Hydrate B has characteristic peaks in the powder X-ray diffraction pattern at values of two theta (° 2θ) of 5.9, 7.8, 8.6, 9.1, 9.5, 10, 10.9, 11.9, 13.2, 13.5, 14.8, 15.7, 16.2, 17.2, 17.7, 18.3, 19, and 19.6.

More preferably, Hydrate B has the PXRD peaks listed in Table 1.

TABLE 1
PXRD Peak of Hydrate B (Form I)

| Peak Position (° 2θ) | Relative Intensity |
|---|---|
| 5.921 | 13.5 |
| 7.802 | 30.8 |
| 8.619 | 35.8 |
| 9.138 | 38 |
| 9.519 | 19.9 |
| 10.019 | 20.3 |
| 10.882 | 63.1 |
| 11.919 | 65.3 |
| 13.201 | 21.9 |
| 13.54 | 40.8 |
| 14.781 | 33.5 |
| 15.681 | 100 |
| 16.163 | 24.7 |
| 17.22 | 50.7 |
| 17.681 | 96 |
| 18.34 | 30.7 |
| 18.96 | 27.1 |
| 19.6 | 34.5 |
| 20.196 | 19.9 |
| 20.902 | 31 |
| 21.16 | 64.6 |

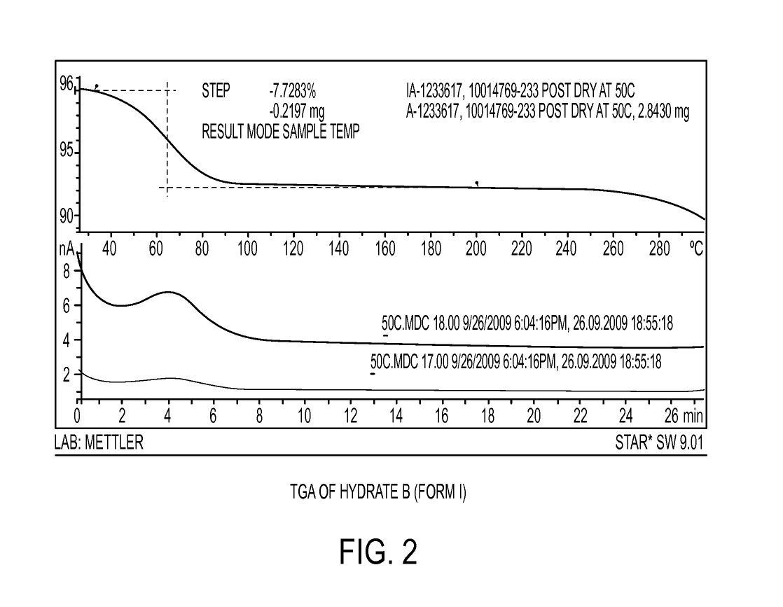

TGA-MS curve displaying weight loss upon heating of Hydrate B is shown in FIG. 2.

In another aspect, the present invention features processes of using Hydrate B to prepare a composition comprising Compound I. In one embodiment, the processes comprise dissolving Hydrate B in a solvent. The solution thus produced can be further dried (e.g., via spray drying or other solvent evaporization techniques). In another embodiment, the processes comprise melting Hydrate B and one or more hydrophilic polymer and optionally one or more pharmaceutically acceptable surfactants. The melt thus prepared can be further solidified (e.g., via melt extrusion).

Preferably, a hydrophilic polymer employed in the present invention has a T_{g} of at least 50° C., more preferably at least 60° C., and highly preferably at least 80° C. including, but not limited to from, 80° C. to 180° C., or from 100° C. to 150° C. Methods for determining T_{g} values of organic polymers are described in INTRODUCTION TO PHYSICAL POLYMER SCIENCE (2nd Edition by L. H. Sperling, published by John Wiley & Sons, Inc., 1992). The T_{g} value can be calculated as the weighted sum of the T_{g} values for homopolymers derived from each of the individual monomers, i.e., the polymer T_{g}=Σ Wᵢ·Xᵢ where Wᵢ is the weight percent of monomer i in the organic polymer, and Xᵢ is the T_{g} value for the homopolymer derived from monomer i. T_{g} values for the homopolymers may be taken from POLYMER HANDBOOK (2nd Edition by J. Brandrup and E. H. Immergut, Editors, published by John Wiley & Sons, Inc., 1975). Hydrophilic polymers with a T_{g} as described above may allow for the preparation of solid dispersions that are mechanically stable and, within ordinary temperature ranges, sufficiently temperature stable so that the solid dispersions may be used as dosage forms without further processing or be compacted to tablets with only a small amount of tabletting aids. Hydrophilic polymers having a T_{g} of below 50° C. may also be used.

Preferably, a hydrophilic polymer employed in the present invention is water-soluble. A solid composition of the present invention can also comprise poorly water-soluble or water-insoluble polymer or polymers, such as cross-linked polymers. A hydrophilic polymer comprised in a solid composition of the present invention preferably has an apparent viscosity, when dissolved at 20° C. in an aqueous solution at 2% (w/v), of 1 to 5000 mPa·s., and more preferably of 1 to 700 mPa·s, and most preferably of 5 to 100 mPa·s.

Hydrophilic polymers suitable for use in a solid composition of the invention include, but are not limited to, homopolymers or copolymers of N-vinyl lactams, such as homopolymers or copolymers of N-vinyl pyrrolidone (e.g., polyvinylpyrrolidone (PVP), or copolymers of N-vinyl pyrrolidone and vinyl acetate or vinyl propionate); cellulose esters or cellulose ethers, such as alkylcelluloses (e.g., methylcellulose or ethylcellulose), hydroxyalkylcelluloses (e.g., hydroxypropylcellulose), hydroxyalkylalkylcelluloses (e.g., hydroxypropylmethylcellulose), and cellulose phthalates or succinates (e.g., cellulose acetate phthalate and hydroxypropylmethylcellulose phthalate, hydroxypropylmethylcellulose succinate, or hydroxypropylmethylcellulose acetate succinate); high molecular polyalkylene oxides, such as polyethylene oxide, polypropylene oxide, and copolymers of ethylene oxide and propylene oxide; polyacrylates or polymethacrylates, such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), and poly(hydroxyalkyl methacrylates); polyacrylamides; vinyl acetate polymers, such as copolymers of vinyl acetate and crotonic acid, and partially hydrolyzed polyvinyl acetate (also referred to as partially saponified “polyvinyl alcohol”); polyvinyl alcohol; oligo- or polysaccharides, such as carrageenans, galactomannans, and xanthan gum; polyhydroxyalkylacrylates; polyhydroxyalkyl-methacrylates; copolymers of methyl methacrylate and acrylic acid; polyethylene glycols (PEGs); or any mixture thereof.

Non-limiting examples of preferred hydrophilic polymers for the invention include polyvinylpyrrolidone (PVP) K17, PVP K25, PVP K30, PVP K90, hydroxypropyl methylcellulose (HPMC) E3, HPMC E5, HPMC E6, HPMC E15, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, HPMC P 55, Ethocel 4, Ethocel 7, Ethocel 10, Ethocel 14, Ethocel 20, copovidone (vinylpyrrolidone-vinyl acetate copolymer 60/40), polyvinyl acetate, methacrylate/methacrylic acid copolymer (Eudragit) L100-55, Eudragit L100, Eudragit S100, polyethylene glycol (PEG) 400, PEG 600, PEG 1450, PEG 3350, PEG 4000, PEG 6000, PEG 8000, poloxamer 124, poloxamer 188, poloxamer 237, poloxamer 338, and poloxamer 407.

Of these, homopolymers or copolymers of N-vinyl pyrrolidone, such as copolymers of N-vinyl pyrrolidone and vinyl acetate, are preferred. A non-limiting example of a preferred polymer is a copolymer of 60% by weight of N-vinyl pyrrolidone and 40% by weight of vinyl acetate. Other preferred polymers include, without limitation, hydroxypropyl methylcellulose (HPMC, also known as hypromellose in USP), such as hydroxypropyl methylcellulose grade E5 (HPMC-E5); and hydroxypropyl methylcellulose acetate succinate (HPMC-AS).

A pharmaceutically acceptable surfactant employed in the present invention is preferably a non-ionic surfactant. More preferably, a solid composition of the present invention comprises a pharmaceutically acceptable surfactant having an HLB value of from 2-20. A solid composition of the present invention can also include a mixture of pharmaceutically acceptable surfactants, with at least one surfactant having an HLB value of no less than 10 and at least another surfactant having an HLB value of below 10. In one example, each surfactant comprised in a solid composition of the invention has an HLB value of at least 10. In another example, each surfactant comprised in a solid composition of the invention has an HLB value of below 10. In yet another example, a solid composition of the present invention includes at least two pharmaceutically acceptable surfactants, one having an HLB value of at least 10 and the other having an HLB value of below 10. The HLB system (Fiedler, H. B., ENCYLOPEDIA OF EXCIPIENTS, 5^{th} ed., Aulendorf: ECV-Editio-Cantor-Verlag (2002)) attributes numeric values to surfactants, with lipophilic substances receiving lower HLB values and hydrophilic substances receiving higher HLB values.

Non-limiting examples of pharmaceutically acceptable surfactants that are suitable for the present invention include polyoxyethylene castor oil derivates, e.g. polyoxyethyleneglycerol triricinoleate or polyoxyl 35 castor oil (Cremophor® EL; BASF Corp.) or polyoxyethyleneglycerol oxystearate such as polyethylenglycol 40 hydrogenated castor oil (Cremophor® RH 40, also known as polyoxyl 40 hydrogenated castor oil or macrogolglycerol hydroxystearate) or polyethylenglycol 60 hydrogenated castor oil (Cremophor® RH 60); or a mono fatty acid ester of polyoxyethylene sorbitan, such as a mono fatty acid ester of polyoxyethylene (20) sorbitan, e.g. polyoxyethylene (20) sorbitan monooleate (Tween® 80), polyoxyethylene (20) sorbitan monostearate (Tween® 60), polyoxyethylene (20) sorbitan monopalmitate (Tween® 40), or polyoxyethylene (20) sorbitan monolaurate (Tween® 20). Other non-limiting examples of suitable surfactants include polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; polyoxyethylene alkylaryl ethers, e.g. polyoxyethylene (2) nonylphenyl ether, polyoxyethylene (3) nonylphenyl ether, polyoxyethylene (4) nonylphenyl ether, polyoxyethylene (3) octylphenyl ether; polyethylene glycol fatty acid esters, e.g. PEG-200 monolaurate, PEG-200 dilaurate, PEG-300 dilaurate, PEG-400 dilaurate, PEG-300 distearate, PEG-300 dioleate; alkylene glycol fatty acid mono esters, e.g. propylene glycol monolaurate (Lauroglycol®); sucrose fatty acid esters, e.g. sucrose monostearate, sucrose distearate, sucrose monolaurate, sucrose dilaurate; sorbitan fatty acid mono esters such as sorbitan mono laurate (Span® 20), sorbitan monooleate, sorbitan monopalnitate (Span® 40), or sorbitan stearate. Other suitable surfactants include, but are not limited to, block copolymers of ethylene oxide and propylene oxide, also known as polyoxyethylene polyoxypropylene block copolymers or polyoxyethylene polypropyleneglycol, such as Poloxamer® 124, Poloxamer® 188, Poloxamer® 237, Poloxamer® 388, or Poloxamer® 407 (BASF Wyandotte Corp.). As described above, a mixture of surfactants can be used in a solid composition of the present invention.

Non-limiting examples of preferred surfactants for the invention include to polysorbate 20, polysorbate 40, polysorbate 60, polysorbate 80, Cremophor RH 40, Cremophor EL, Gelucire 44/14, Gelucire 50/13, D-alpha-tocopheryl polyethylene glycol 1000 succinate (vitamin E TPGS), propylene glycol laurate, sodium lauryl sulfate, and sorbitan monolaurate.

In one embodiment, the hydrophilic polymer can be selected, for example, from the group consisting of homopolymer of N-vinyl lactam, copolymer of N-vinyl lactam, cellulose ester, cellulose ether, polyalkylene oxide, polyacrylate, polymethacrylate, polyacrylamide, polyvinyl alcohol, vinyl acetate polymer, oligosaccharide, and polysaccharide. As a non-limiting example, the hydrophilic polymer is selected from the group consisting of homopolymer of N-vinyl pyrrolidone, copolymer of N-vinyl pyrrolidone, copolymer of N-vinyl pyrrolidone and vinyl acetate, copolymer of N-vinyl pyrrolidone and vinyl propionate, polyvinylpyrrolidone, methylcellulose, ethylcellulose, hydroxyalkylcelluloses, hydroxypropylcellulose, hydroxyalkylalkylcellulose, hydroxypropylmethylcellulose, cellulose phthalate, cellulose succinate, cellulose acetate phthalate, hydroxypropylmethylcellulose phthalate, hydroxypropylmethylcellulose succinate, hydroxypropylmethylcellulose acetate succinate, polyethylene oxide, polypropylene oxide, copolymer of ethylene oxide and propylene oxide, methacrylic acid/ethyl acrylate copolymer, methacrylic acid/methyl methacrylate copolymer, butyl methacrylate/2-dimethylaminoethyl methacrylate copolymer, poly(hydroxyalkyl acrylate), poly(hydroxyalkyl methacrylate), copolymer of vinyl acetate and crotonic acid, partially hydrolyzed polyvinyl acetate, carrageenan, galactomannan, and xanthan gum. Preferably, the hydrophilic polymer is selected from polyvinylpyrrolidone (PVP) K17, PVP K25, PVP K30, PVP K90, hydroxypropyl methylcellulose (HPMC) E3, HPMC E5, HPMC E6, HPMC E15, HPMC K3, HPMC A4, HPMC A15, HPMC acetate succinate (AS) LF, HPMC AS MF, HPMC AS HF, HPMC AS LG, HPMC AS MG, HPMC AS HG, HPMC phthalate (P) 50, HPMC P 55, Ethocel 4, Ethocel 7, Ethocel 10, Ethocel 14, Ethocel 20, copovidone (vinylpyrrolidone-vinyl acetate copolymer 60/40), polyvinyl acetate, methacrylate/methacrylic acid copolymer (Eudragit) L100-55, Eudragit L100, Eudragit S100, polyethylene glycol (PEG) 400, PEG 600, PEG 1450, PEG 3350, PEG 4000, PEG 6000, PEG 8000, poloxamer 124, poloxamer 188, poloxamer 237, poloxamer 338, or poloxamer 407. More preferably, the hydrophilic polymer is selected from homopolymers of vinylpyrrolidone (e.g., PVP with Fikentscher K values of from 12 to 100, or PVP with Fikentscher K values of from 17 to 30), or copolymers of 30 to 70% by weight of N-vinylpyrrolidone (VP) and 70 to 30% by weight of vinyl acetate (VA) (e.g., a copolymer of 60% by weight VP and 40% by weight VA). The surfactant can be selected, for example, from the group consisting of polyoxyethyleneglycerol triricinoleate or polyoxyl 35 castor oil (Cremophor® EL; BASF Corp.) or polyoxyethyleneglycerol oxystearate, mono fatty acid ester of polyoxyethylene sorbitan, polyoxyethylene alkyl ether, polyoxyethylene alkylaryl ether, polyethylene glycol fatty acid ester, alkylene glycol fatty acid mono ester, sucrose fatty acid ester, and sorbitan fatty acid mono ester. As a non-limited example, the surfactant is selected from the group consisting of polyethylenglycol 40 hydrogenated castor oil (Cremophor® RH 40, also known as polyoxyl 40 hydrogenated castor oil or macrogolglycerol hydroxystearate), polyethylenglycol 60 hydrogenated castor oil (Cremophor® RH 60), a mono fatty acid ester of polyoxyethylene (20) sorbitan (e.g. polyoxyethylene (20) sorbitan monooleate (Tween® 80), polyoxyethylene (20) sorbitan monostearate (Tween® 60), polyoxyethylene (20) sorbitan monopalmitate (Tween® 40), or polyoxyethylene (20) sorbitan monolaurate (Tween® 20)), polyoxyethylene (3) lauryl ether, polyoxyethylene (5) cetyl ether, polyoxyethylene (2) stearyl ether, polyoxyethylene (5) stearyl ether, polyoxyethylene (2) nonylphenyl ether, polyoxyethylene (3) nonylphenyl ether, polyoxyethylene (4) nonylphenyl ether, polyoxyethylene (3) octylphenyl ether, PEG-200 monolaurate, PEG-200 dilaurate, PEG-300 dilaurate, PEG-400 dilaurate, PEG-300 distearate, PEG-300 dioleate, propylene glycol monolaurate, sucrose monostearate, sucrose distearate, sucrose monolaurate, sucrose dilaurate, sorbitan monolaurate, sorbitan monooleate, sorbitan monopalnitate, and sorbitan stearate. Preferably, the surfactant is selected from polysorbate 20, polysorbate 40, polysorbate 60, polysorbate 80, Cremophor RH 40, Cremophor EL, Gelucire 44/14, Gelucire 50/13, D-alpha-tocopheryl polyethylene glycol 1000 succinate (vitamin E TPGS), propylene glycol laurate, sodium lauryl sulfate, or sorbitan monolaurate. More preferably, the surfactant is selected from sorbitan monolaurate or D-alpha-tocopheryl polyethylene glycol 1000 succinate.

In another embodiment, the hydrophilic polymer is a homopolymer or copolymer of N-vinyl pyrrolidone (e.g., copovidone). The pharmaceutically acceptable surfactant can be, e.g., vitamin E TPGS, or sorbitan monolaurate.

Compound I can be prepared according to the procedures described in U.S. Provisional Patent Application No. 61/243,596, filed Sep. 18, 2009. To a mixture of (5)-2-(methoxycarbonylamino)-3-methylbutanoic acid (19.66 g, 112 mmol) and N-hydroxysuccinimide (13.29 g, 116 mmol) was added ethyl acetate (250 ml), and the mixture was cooled to 0-5° C. Diisopropylcarbodiimide (13.88 g, 110 mmol) was added and the reaction mixture was stirred at 0-5° C. for about 1 hour. The reaction mixture was warmed to room temperature. The solids (diisopropylurea by-product) were filtered and rinsed with ethyl acetate. The filtrate was concentrated in vacuo to an oil. Isopropyl alcohol (200 ml) was added to the oil and the mixture was heated to about 50° C. to obtain a homogeneous solution. Upon cooling, crystalline solids formed. The solids were filtered and washed with isopropyl alcohol (3×20 ml) and dried to give (S)-2,5-dioxopyrrolidin-1-yl 2-(methoxycarbonylamino)-3-methylbutanoate as a white solid (23.2 g, 77% yield).

To a mixture of L-proline (4.44 g, 38.6 mmol), water (20 ml), acetonitrile (20 ml) and DIEA (9.5 g, 73.5 mmol) was added a solution of (S)-2,5-dioxopyrrolidin-1-yl 2-(methoxycarbonylamino)-3-methylbutanoate (10 g, 36.7 mmol) in acetonitrile (20 mL) over 10 minutes. The reaction mixture was stirred overnight at room temperature. The solution was concentrated under vacuum to remove the acetonitrile. To the resulting clear water solution was added 6N HCl (9 ml) until pH˜2. The solution was transferred to a separatory funnel and 25% NaCl (10 ml) was added and the mixture was extracted with ethyl acetate (75 ml), and then again with ethyl acetate (6×20 ml), and the combined extracts were washed with 25% NaCl (2×10 ml). The solvent was evaporated to give a thick oil. Heptane was added and the solvent was evaporated to give a foam, which was dried under high vacuum. Diethyl ether was added and the solvent was evaporated to give a foam, which was dried under high vacuum to give (S)-1-((S)-2-(methoxycarbonylamino)-3-methylbutanoyl)pyrrolidine-2-carboxylic acid (10.67 g) as a white solid.

(S)-1-((S)-2-(methoxycarbonylamino)-3-methylbutanoyl)pyrrolidine-2-carboxylic acid can also be prepared according to the following procedure. To a flask was charged L-valine (35 g, 299 mmol), 1N sodium hydroxide solution (526 ml, 526 mmol) and sodium carbonate (17.42 g, 164 mmol). The mixture was stirred for 15 min to dissolve solids and then cooled to 15° C. Methyl chloroformate (29.6 g, 314 mmol) was added slowly to the reaction mixture. The mixture was then stirred at rt for 30 min. The mixture was cooled to 15° C. and pH adjusted to ˜5.0 with concentrated HCl solution. 100 mL of 2-methytetrahydrofuran (2-MeTHF) was added and the adjustment of pH continued until the pH reached ˜2.0. 150 mL of 2-MeTHF was added and the mixture was stirred for 15 min. Layers were separated and the aqueous layer extracted with 100 mL of 2-MeTHF. The combined organic layer was dried over anhyd Na₂SO₄ and filtered, and Na₂SO₄ cake was washed with 50 mL of 2-MeTHF. The product solution was concentrated to ˜100 mL, chased with 120 mL of IPAc twice. 250 mL of heptanes was charged slowly and then the volume of the mixture was concentrated to 300 mL. The mixture was heated to 45° C. and 160 mL of heptanes charged. The mixture was cooled to rt in 2 h, stirred for 30 min, filtered and washed with 2-MeTHF/heptanes mixture (1:7, 80 mL). The wetcake was dried at 55° C. for 24 h to give 47.1 g of Moc-L-Val-OH product as a white solid (90%). Moc-L-Val-OH (150 g, 856 mmol), HOBt hydrate (138 g, 899 mmol) and DMF (1500 ml) were charged to a flask. The mixture was stirred for 15 min to give a clear solution. EDC hydrochloride (172 g, 899 mmol) was charged and mixed for 20 min. The mixture was cooled to 13° C. and (L)-proline benzyl ester hydrochloride (207 g, 856 mmol) charged. Triethylamine (109 g, 1079 mmol) was then charged in 30 min. The resulting suspension was mixed at rt for 1.5 h. The reaction mixture was cooled to 15° C. and 1500 mL of 6.7% NaHCO₃ charged in 1.5 h, followed by the addition of 1200 mL of water over 60 min. The mixture was stirred at rt for 30 min, filtered and washed with water/DMF mixture (1:2, 250 mL) and then with water (1500 mL). The wetcake was dried at 55° C. for 24 h to give 282 g of product as a white solid (90%). The resulting solids (40 g) and 5% Pd/Alumina were charged to a Parr reactor followed by THF (160 mL). The reactor was sealed and purged with nitrogen (6×20 psig) followed by a hydrogen purge (6×30 psig). The reactor was pressurized to 30 psig with hydrogen and agitated at room temperature for approximately 15 hours. The resulting slurry was filtered through a GF/F filter and concentrated to approximately 135 g solution. Heptane was added (120 mL), and the solution was stirred until solids formed. After an addition 2-3 hours additional heptane was added drop-wise (240 mL), the slurry was stirred for approximately 1 hour, then filtered. The solids were dried to afford (S)-1-((S)-2-(methoxycarbonylamino)-3-methylbutanoyl)pyrrolidine-2-carboxylic acid.

Anhydrous zinc(II) chloride (2.73 g, 20.00 mmol) was stirred in dry benzene (15 ml) while diethylamine (1.558 ml, 15.00 mmol) and t-butanol (1.435 ml, 15.00 mmol) were added, and the resulting mixture was stirred at room temperature for 90 min to give a cloudy solution. To this mixture was added 2-bromo-1-(4-nitrophenyl)ethanone (2.44 g, 10.00 mmol) and 1-(4-nitrophenyl)ethanone (2.477 g, 15.00 mmol), and the resulting mixture was stirred at room temperature overnight. The mixture was poured into water (50 mL) and extracted with ethyl acetate (3×50 ml). The combined organic layers were dried over Na₂SO₄, filtered and concentrated. The resulting residue was triturated with dichloromethane to give an orange solid that was collected by filtration and dried to give 1,4-Bis(4-nitrophenyl)butane-1,4-dione (2.0 gm, 61% yield).

To (S)-(−)-α,α-diphenyl-2-pyrrolidinemethanol (2.71 g, 10.70 mmol) was added THF (80 mL) at 23° C. The very thin suspension was treated with trimethyl borate (1.44 g, 13.86 mmol) over 30 seconds, and the resulting solution was mixed at 23° C. for 1 h. The solution was cooled to 16-19° C., and N,N-diethylaniline borane (21.45 g, 132 mmol) was added dropwise via syringe over 3-5 min (caution: vigorous H₂ evolution), while the internal temperature was maintained at 16-19° C. After 15 min, the H₂ evolution had ceased. To a separate vessel was added 1,4-Bis(4-nitrophenyl)butane-1,4-dione (22.04 g, 95 wt %, 63.8 mmol), followed by THF (80 mL), to form an orange slurry. After cooling the slurry to 11° C., the borane solution was transferred via cannula into the dione slurry over 3-5 min. During this period, the internal temperature of the slurry rose to 16° C. After the addition was complete, the reaction was maintained at 20-27° C. for an additional 2.5 h. After reaction completion, the mixture was cooled to 5° C. and methanol (16.7 g, 521 mmol) was added dropwise over 5-10 min, maintaining an internal temperature <20° C. (note: vigorous H₂ evolution). After the exotherm had ceased (ca. 10 min), the temperature was adjusted to 23° C., and the reaction was mixed until complete dissolution of the solids had occurred. Ethyl acetate (300 mL) and 1 M HCl (120 mL) were added, and the phases were partitioned. The organic phase was then washed successively with 1 M HCl (2×120 mL), H₂O (65 mL), and 10% aq. NaCl (65 mL). The organics were dried over MgSO₄, filtered, and concentrated in vacuo. Crystallization of the product occurred during the concentration. The slurry was warmed to 50° C., and heptane (250 mL) was added over 15 min. The slurry was then allowed to mix at 23° C. for 30 min and filtered. The wet cake was washed with 3:1 heptane:ethyl acetate (75 mL), and the orange, crystalline solids were dried at 45° C. for 24 h to provide (1R,4R)-1,4-bis(4-nitrophenyl)butane-1,4-diol (15.35 g, 99.3% ee, 61% yield), which was contaminated with 11% of the meso isomer (vs. dl isomer).

(1R,4R)-1,4-bis(4-nitrophenyl)butane-1,4-diol (5.01 g, 13.39 mmol) was combined with 2-methyltetrahydrofuran (70 mL) and cooled to −5° C., and N,N-diisopropylethylamine (6.81 g, 52.7 mmol) was added over 30 seconds. Separately, a solution of methanesulfonic anhydride (6.01 g, 34.5 mmol) in 2-methyltetrahydrofuran (30 mL) was prepared and added to the diol slurry over 3 min., maintaining the internal temperature between −15° C. and −25° C. After mixing for 5 min at −15° C., the cooling bath was removed and the reaction was allowed to warm slowly to 23° C. and mixed for 30 minutes. After reaction completion, the crude slurry ((1R,4R)-1,4-bis(4-nitrophenyl)butane-1,4-diyl dimethanesulfonate) was carried immediately into the next step.

To the crude product solution (1R,4R)-1,4-bis(4-nitrophenyl)butane-1,4-diyl dimethanesulfonate (7.35 g, 13.39 mmol) was added 4-tert-butylaniline (13.4 g, 90 mmol) at 23° C. over 1 minute. The reaction was heated to 65° C. for 2 h. After completion, the reaction mixture was cooled to 23° C. and diluted with 2-methyltetrahydrofuran (100 mL) and 1 M HCl (150 mL). After partitioning the phases, the organic phase was treated with 1 M HCl (140 mL), 2-methyltetrahydrofuran (50 mL), and 25 wt % aq. NaCl (100 mL), and the phases were partitioned. The organic phase was washed with 25 wt % aq. NaCl (50 mL), dried over MgSO₄, filtered, and concentrated in vacuo to approximately 20 mL. Heptane (30 mL) and additional 2-methyltetrahydrofuran were added in order to induce crystallization. The slurry was concentrated further, and additional heptane (40 mL) was slowly added and the slurry was filtered, washing with 2-methyltetrahydrofuran:heptane (1:4, 20 mL). The solids were suspended in MeOH (46 mL) for 3 h, filtered, and the wet solid was washed with additional MeOH (18 mL). The solid was dried at 45° C. in a vacuum oven for 16 h to provide (2S,5S)-1-(4-tert-butylphenyl)-2,5-bis(4-nitrophenyl)pyrrolidine (3.08 g, 51% 2-step yield).

To a 160 ml Parr stirrer hydrogenation vessel was added (2S,5S)-1-(4-tert-butylphenyl)-2,5-bis(4-nitrophenyl)pyrrolidine (2 g, 4.49 mmol), followed by 60 ml of THF, and Raney Nickel Grace 2800 (1 g, 50 wt % (dry basis)) under a stream of nitrogen. The reactor was assembled and purged with nitrogen (8×20 psig) followed by purging with hydrogen (8×30 psig). The reactor was then pressurized to 30 psig with hydrogen and agitation (700 rpm) began and continued for a total of 16 h at room temperature. The slurry was filtered by vacuum filtration using a GF/F Whatman glass fiber filter. Evaporation of the filtrate to afford a slurry followed by the addition heptane and filtration gave the crude 4,4′-((2S,5S)-1-(4-tert-butylphenyl)pyrrolidine-2,5-diyl)dianiline, which was dried and used directly in the next step.

To a solution of 4,4′-((2S,5S)-1-(4-tert-butylphenyl)pyrrolidine-2,5-diyl)dianiline (1.64 g, 4.25 mmol) in DMF (20 ml), (S)-1-((S)-2-(methoxycarbonylamino)-3-methylbutanoyl)pyrrolidine-2-carboxylic acid (2.89 g, 10.63 mmol), and HATU (4.04 g, 10.63 mmol) in DMF (150 mL) was added triethylamine (1.07 g, 10.63 mmol), and the solution was stirred at room temperature for 90 min. To the reaction mixture was poured 20 mL of water, and the white precipitate obtained was filtered, and the solid was washed with water (3×5 mL). The solid was blow dried for 1 h. The crude material was loaded on a silica gel column and eluted with a gradient starting with ethyl acetate/heptane (3/7), and ending with pure ethyl acetate. The desired fractions were combined and solvent distilled off to give a very light yellow solid, which was dried at 45° C. in a vacuum oven with nitrogen purge for 15 h to give Compound I (2.3 g, 61% yield).

Alternately, 4,4′-((2S,5S)-1-(4-tert-butylphenyl)pyrrolidine-2,5-diyl)dianiline (11.7 g, 85 wt %, 25.8 mmol) and (S)-1-((S)-2-(methoxycarbonylamino)-3-methylbutanoyl)pyrrolidine-2-carboxylic acid (15.45 g, 56.7 mmol) are suspended in EtOAc (117 mL), diisopropylethylamine (18.67 g, 144 mmol) is added and the solution is cooled to 0° C. In a separate flask, 1-propanephosphonic acid cyclic anhydride (T3P®) (46.0 g, 50 wt % in EtOAc, 72.2 mmol) was dissolved in EtOAc (58.5 mL), and charged to an addition funnel. The T₃P solution is added to the reaction mixture drop-wise over 3-4 h and stirred until the reaction is complete. The reaction is warmed to room temperature, and washed with 1M HCl/7.5 wt % NaCl (100 mL), then washed with 5% NaHCO₃ (100 mL), then washed with 5% NaCl solution (100 mL). The solution was concentrated to approximately 60 mL, EtOH (300 mL) was added, and the solution was concentrated to 84 g solution.

A portion of the EtOH solution of product (29 g) was heated to 40° C., and added 134 g 40 w % EtOH in H₂O. A slurry of seeds in 58 wt/wt % EtOH/H₂O was added, allowed to stir at 35-40° C. (e.g, at 35 or 40° C.) for several hours, then cooled to 0° C. The slurry was then filtered, and washed with 58 wt/wt % EtOH/H₂O. The product was dried at 40-60° C. (e.g., 40° C.) under vacuum, and then rehydrated by placing a tray of water in the vacuum oven (or in a tray dryer using 40° C. and a humidifed atmosphere) to give Compound I in a hydrate crystalline form (Hydrate B).

The seeds described above was a EtOH—H₂O solvate of Compound I and was originally made according to the following procedure. A solution was first prepared by slurrying the amorphous, solid Compound I in 37 weight % EtOH in heptane at room temperature. The saturated solution (with respect to the amorphous solid) was then filtered over to a new vial to give a clear solution and seeded with partially crystalline solid obtained from the same solvent system. Crystallization took place within a few hours to produce the “Anhydrate A” form of Compound I. Then a solution of 60 w % EtOH in H₂O at 5° C. was prepared using the amorphous Compound I. To this clear solution was added seeds of mainly Anhydrate A and desolvated EtOH solvate. Conversion to the EtOH—H₂O solvate took place within 2 days to produce the original seeds, which can be used to make additional seeds.

Hydrate B can be processes by a variety of techniques such as, without limitation, melt-extrusion, spray-drying, co-precipitation, freeze drying, or other solvent evaporation techniques, with melt-extrusion and spray-drying being preferred. The melt-extrusion process typically comprises the steps of preparing a melt which includes the active ingredient(s), the hydrophilic polymer(s) and preferably the surfactant(s), and then cooling the melt until it solidifies. “Melting” means a transition into a liquid or rubbery state in which it is possible for one component to get embedded, preferably homogeneously embedded, in the other component or components. In many cases, the polymer component(s) will melt and the other components including the active ingredient(s) and surfactant(s) will dissolve in the melt thereby forming a solution. Melting usually involves heating above the softening point of the polymer(s). The preparation of the melt can take place in a variety of ways. The mixing of the components can take place before, during or after the formation of the melt. For example, the components can be mixed first and then melted or be simultaneously mixed and melted. The melt can also be homogenized in order to disperse the active ingredient(s) efficiently. In addition, it may be convenient first to melt the polymer(s) and then to mix in and homogenize the active ingredient(s). In one example, all materials except surfactant(s) are blended and fed into an extruder, while the surfactant(s) is molten externally and pumped in during extrusion.

In another example, the melt comprises Compound I and one or more hydrophilic polymers described above, and the melt temperature is in the range of from 100 to 170° C., preferably from 120 to 150° C., and highly preferably from 135 to 140° C. The melt can also include a pharmaceutically acceptable surfactant described above.

In still another example, the melt comprises Compound I, at least another anti-HCV agent described above, and one or more hydrophilic polymers described above. The melt can also include a pharmaceutically acceptable surfactant described above.

To start a melt-extrusion process, the active ingredient(s) (e.g., Compound I, or a combination of Compound I and at least another anti-HCV agent) can be employed in their solid forms, such as their respective crystalline forms. The active ingredient(s) can also be employed as a solution or dispersion in a suitable liquid solvent such as alcohols, aliphatic hydrocarbons, esters or, in some cases, liquid carbon dioxide. The solvent can be removed, e.g. evaporated, upon preparation of the melt.

Various additives can also be included in the melt, for example, flow regulators (e.g., colloidal silica), binders, lubricants, fillers, disintegrants, plasticizers, colorants, or stabilizers (e.g., antioxidants, light stabilizers, radical scavengers, and stabilizers against microbial attack).

The melting and/or mixing can take place in an apparatus customary for this purpose. Particularly suitable ones are extruders or kneaders. Suitable extruders include single screw extruders, intermeshing screw extruders or multiscrew extruders, preferably twin screw extruders, which can be corotating or counterrotating and, optionally, be equipped with kneading disks. It will be appreciated that the working temperatures will be determined by the kind of extruder or the kind of configuration within the extruder that is used. Part of the energy needed to melt, mix and dissolve the components in the extruder can be provided by heating elements. However, the friction and shearing of the material in the extruder may also provide a substantial amount of energy to the mixture and aid in the formation of a homogeneous melt of the components.

The melt can range from thin to pasty to viscous. Shaping of the extrudate can be conveniently carried out by a calender with two counter-rotating rollers with mutually matching depressions on their surface. The extrudate can be cooled and allow to solidify. The extrudate can also be cut into pieces, either before (hot-cut) or after solidification (cold-cut).

The solidified extrusion product can be further milled, ground or otherwise reduced to granules. The solidified extrudate, as well as each granule produced, comprises a solid dispersion, preferably a solid solution, of the active ingredient(s) in a matrix comprised of the hydrophilic polymer(s) and optionally the pharmaceutically acceptable surfactant(s). Where the granules do not contain any surfactant, a pharmaceutically acceptable surfactant described above can be added to and blended with the granules. The extrusion product can also be blended with other active ingredient(s) and/or additive(s) before being milled or ground to granules. The granules can be further processed into suitable solid oral dosage forms.

In one example, copovidone and one or more surfactants are mixed and granulated, followed by the addition of aerosil and Compound I. The mixture, which may contain for example 5% by weight of Compound I, is then milled. The mixture is then subject to extrusion, and the extrudate thus produced can be milled and sieved for further processing to make capsules or tablets. Surfactant(s) employed in this example can also be added through liquid dosing during extrusion.

The approach of solvent evaporation, via spray-drying, provides the advantage of allowing for processability at lower temperatures, if needed, and allows for other modifications to the process in order to further improve powder properties. The spray-dried powder can then be formulated further, if needed, and final drug product is flexible with regards to whether capsule, tablet or any other solid dosage form is desired.

Exemplary spray-drying processes and spray-drying equipment are described in K. Masters, SPRAY DRYING HANDBOOK (Halstead Press, New York, 4^{th} ed., 1985). Non-limiting examples of spray-drying devices that are suitable for the present invention include spray dryers manufactured by Niro Inc. or GEA Process Engineering Inc., Buchi Labortechnik AG, and Spray Drying Systems, Inc. A spray-drying process generally involves breaking up a liquid mixture into small droplets and rapidly removing solvent from the droplets in a container (spray drying apparatus) where there is a strong driving force for evaporation of solvent from the droplets. Atomization techniques include, for example, two-fluid or pressure nozzles, or rotary atomizers. The strong driving force for solvent evaporation can be provided, for example, by maintaining the partial pressure of solvent in the spray drying apparatus well below the vapor pressure of the solvent at the temperatures of the drying droplets. This may be accomplished by either (1) maintaining the pressure in the spray drying apparatus at a partial vacuum; (2) mixing the liquid droplets with a warm drying gas (e.g., heated nitrogen); or (3) both.

The temperature and flow rate of the drying gas, as well as the spray dryer design, can be selected so that the droplets are dry enough by the time they reach the wall of the apparatus. This help to ensure that the dried droplets are essentially solid and can form a fine powder and do not stick to the apparatus wall. The spray-dried product can be collected by removing the material manually, pneumatically, mechanically or by other suitable means. The actual length of time to achieve the preferred level of dryness depends on the size of the droplets, the formulation, and spray dryer operation. Following the solidification, the solid powder may stay in the spray drying chamber for additional time (e.g., 5-60 seconds) to further evaporate solvent from the solid powder. The final solvent content in the solid dispersion as it exits the dryer is preferably at a sufficiently low level so as to improve the stability of the final product. For instance, the residual solvent content of the spray-dried powder can be less than 2% by weight. Highly preferably, the residual solvent content is within the limits set forth in the International Conference on Harmonization (ICH) Guidelines. In addition, it may be useful to subject the spray-dried composition to further drying to lower the residual solvent to even lower levels. Methods to further lower solvent levels include, but are not limited to, fluid bed drying, infra-red drying, tumble drying, vacuum drying, and combinations of these and other processes.

Like the solid extrudate described above, the spray dried product contains a solid dispersion, preferably a solid solution, of the active ingredient(s) in a matrix comprised of the hydrophilic polymer(s) and optionally the pharmaceutically acceptable surfactant(s). Where the spray dried product does not contain any surfactant, a pharmaceutically acceptable surfactant described above can be added to and blended with the spray-dried product before further processing.

Before feeding into a spray dryer, the active ingredient(s) (e.g., Compound I, or a combination of Compound I and at least another anti-HCV agent), the hydrophilic polymer(s), as well as other optional active ingredients or excipients such as the pharmaceutically acceptable surfactant(s), can be dissolved in a solvent. Suitable solvents include, but are not limited to, alkanols (e.g., methanol, ethanol, 1-propanol, 2-propanol or mixtures thereof), acetone, acetone/water, alkanol/water mixtures (e.g., ethanol/water mixtures), or combinations thereof. The solution can also be preheated before being fed into the spray dryer.

The solid dispersion produced by melt-extrusion, spray-drying or other techniques can be prepared into any suitable solid oral dosage forms. In one embodiment, the solid dispersion prepared by melt-extrusion, spray-drying or other techniques (e.g., the extrudate or the spray-dried powder) can be compressed into tablets. The solid dispersion can be either directly compressed, or milled or ground to granules or powders before compression. Compression can be done in a tablet press, such as in a steel die between two moving punches. When a solid composition of the present invention comprises Compound I and another anti-HCV agent, it is possible to separately prepare solid dispersions of each individual active ingredient and then blend the optionally milled or ground solid dispersions before compacting. Compound I and other active ingredient(s) can also be prepared in the same solid dispersion, optionally milled and/or blended with other additives, and then compressed into tablets.

At least one additive selected from flow regulators, binders, lubricants, fillers, disintegrants, or plasticizers may be used in compressing the solid dispersion. These additives can be mixed with ground or milled solid dispersion before compacting. Disintegrants promote a rapid disintegration of the compact in the stomach and keeps the liberated granules separate from one another. Non-limiting examples of suitable disintegrants are cross-linked polymers such as cross-linked polyvinyl pyrrolidone, cross-linked sodium carboxymethylcellulose or sodium croscarmellose. Non-limiting examples of suitable fillers (also referred to as bulking agents) are lactose monohydrate, calcium hydrogenphosphate, microcrystalline cellulose (e.g., Avicell), silicates, in particular silicium dioxide, magnesium oxide, talc, potato or corn starch, isomalt, or polyvinyl alcohol. Non-limiting examples of suitable flow regulators include highly dispersed silica (e.g., colloidal silica such as Aerosil), and animal or vegetable fats or waxes. Non-limiting examples of suitable lubricants include polyethylene glycol (e.g., having a molecular weight of from 1000 to 6000), magnesium and calcium stearates, sodium stearyl fumarate, and the like.

Various other additives may also be used in preparing a solid composition of the present invention, for example dyes such as azo dyes, organic or inorganic pigments such as aluminium oxide or titanium dioxide, or dyes of natural origin; stabilizers such as antioxidants, light stabilizers, radical scavengers, stabilizers against microbial attack.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. Hydrate B of Compound I.
